(19) United States
(12) Patent Application Publication
(10) Pub. No.: US 2022/0119892 A1
(43) Pub. Date: Apr. 21, 2022

(54) METHODS FOR CANCER SCREENING AND MONITORING BY CANCER MASTER REGULATORS MARKERS IN LIQUID BIOPSY

(71) Applicant: University of Florida Research Foundation, Incorporated, Gainesville, FL (US)

(72) Inventors: David Tran, Gainesville, FL (US); Son Bang Le, Gainesville, FL (US)

(21) Appl. No.: 17/428,475

(22) PCT Filed: Feb. 6, 2020
(86) PCT No.: PCT/US2020/017062

§ 371 Date: Aug. 4, 2021

Related U.S. Application Data
(60) Provisional application No. 62/802,620, filed on Feb. 7, 2019.

(51) Int. Cl. C12Q 1/6886

(52) U.S. Cl. CPC C12Q 1/6886; C12Q 2600/154; C12Q 2600/118

(57) ABSTRACT

Provided herein are methods of assessing, detecting, monitoring the presence, or monitoring progression of cancer in a subject, or assessing or predicting prognosis or survival of a subject having cancer. Also provided are methods of measuring chromosomal accessibility of the chromosomal locus of cancer master regulator genes or downstream genes in the master regulator network, methods of measuring chromosomal DNA methylation at the chromosomal locus of cancer master regulator genes or downstream genes, and methods measuring chromosomal DNA methylation at the chromosomal locus of the promoter and/or regulatory regions of cancer master regulator genes or downstream genes.

### BACKGROUND

Current cancer screening using liquid biopsy relies on massive deep genomic sequencing to detect rare cancer-cell-derived genetic materials in bodily fluids. This process is costly and fraught with high false negative (FN) and false positive (FP) rates. The high FN rate is due to rare mutations accounting for a sizable share of cancer patients. The high FP rate is due to low penetrance of many cancer-associated mutations. Early detection of cancer in patients without obvious clinical evidence of cancer is important, and there remains a need for easy methods of cancer screening in blood.

### SUMMARY

Provided are methods of assessing, detecting, monitoring the presence, or monitoring progression of cancer in a subject, or assessing or predicting prognosis or survival of a subject having cancer. In some embodiments, the methods comprise measuring chromosomal accessibility of the chromosomal locus or one or more cancer master regulator (master regulator) genes or one or more gene downstream of a master regulator in the master regulator network (downstream gene). In some embodiments, the methods comprise measuring chromosomal DNA methylation at the chromosomal locus of one or more master regulator genes or one or more downstream genes. In some embodiments, the methods comprise measuring chromosomal DNA methylation at the chromosomal locus of the promoter and/or regulatory regions of one or more master regulator genes or one or more downstream genes In some embodiments, the methods comprise measuring differential expression of one or more downstream genes.

Measuring chromosomal accessibility or chromosomal DNA methylation can comprise measuring the chromosomal accessibility or chromosomal DNA methylation of a master regulator gene or a gene downstream of the master regulator in the master regulator network in a sample from a subject and comparing the chromosomal accessibility or chromosomal DNA methylation with the chromosomal accessibility or chromosomal DNA methylation of a corresponding gene in a healthy reference sample. Similarly, measuring differential expression of a downstream gene can comprise measuring expression of the downstream gene in a sample from a subject and comparing the expression with the expression of a corresponding gene in a healthy reference sample. An increase in the level of chromosomal accessibility of the master regulator, a decrease in DNA methylation of the master regulator, or differential (increased or decreased) expression of the downstream gene in the subject relative to the level of chromosomal accessibility, DNA methylation, or expression of the corresponding master regulator or downstream gene in the healthy reference sample indicates the possible presence of cancer in the subject, an increase or risk of increase in cancer progression in the subject, a possible increased risk of developing cancer in the subject, a poor prognosis, or decreased predicted survival time for the subject. In some embodiments, chromosomal accessibility or chromosomal DNA methylation is measure is the promotor or regulatory region of a master regulator gene or a gene downstream of the master regulator in the master regulator network. The methods can be used to guide or suggest treatments or changes in treatment of a subject.

In some embodiments, the methods are used to assess whether a subject has a poor survival prognosis for cancer comprising: analyzing the chromosomal accessibility or DNA methylation of least one master regulator in a sample from the subject, wherein an increase in the chromosomal accessibility or DNA methylation level of the at least one master regulator relative to the chromosomal accessibility or DNA methylation level of the corresponding master regulator in a healthy reference sample, is indicative that the subject has a poor survival prognosis for the cancer. The sample can be, but is not limited to, a liquid sample. The liquid sample can be, but is not limited to, a blood sample. Cancer cells, including cancer stem cells, can leave the primary tumor and spread. The methods described herein can be used to detect these migrating cancer cells in blood samples. The cancer can be, but is not limited to, glioblastoma (GBM) and glioblastoma-related cancers. Exemplary cancer master regulators are provided in FIG. 4-7.

In some embodiments, the methods are used to assess whether a subject has a poor survival prognosis for cancer comprising: analyzing expression of least one factor downstream in the master regulator network (downstream factor) in a sample from the subject, wherein an increase in the expression level of the at least one downstream factor relative to the expression level of the corresponding downstream factor in a healthy reference sample, is indicative that the subject has a poor survival prognosis for the cancer. The sample can be, but is not limited to, a liquid sample. The liquid sample can be, but is not limited to, a blood sample. The cancer can be, but is not limited to, glioblastoma and glioblastoma-related cancers. The downstream factor can be a downstream factor differentially expressed in glioblastoma stem cells (GSCs). Exemplary downstream factors are provided in Example 4 and Table 1.

In some embodiments, one or more cancer therapies is administered to a subject identified as having a poor prognosis or decreased survival time.

### BRIEF DESCRIPTION OF THE SEVERAL VIEWS OF THE DRAWING(S)

Having thus described the invention in general terms, reference will now be made to the accompanying drawings, which are not necessarily drawn to scale.

FIG. 1 illustrates the ATAC-seq (Assay for Transposase-Accessible Chromatin using sequence) approach to detect chromosomal accessibility at specific chromosomal locations. Presence or absence of insertion and/or disruption of a gene locus can be detected by loss of the locus as measured by reduced PCR amplification of the locus, or by Sanger sequencing of PCR amplified locus.

FIG. 2 illustrates the ATAC-seq (Assay for Transposase-Accessible Chromatin using sequence) approach to detect chromosomal accessibility at specific chromosomal locations. The transposon contains a phosphate group at the end of the transposon. Presence or absence of insertion and/or disruption of a gene locus can be detected by loss of the locus as measured by reduced PCR amplification of the locus, or by Sanger sequencing of PCR amplified locus.

FIG. 3 illustrates the top master regulators that are differentially expressed in glioblastoma stem cells (GSCs) v. astrocytes.

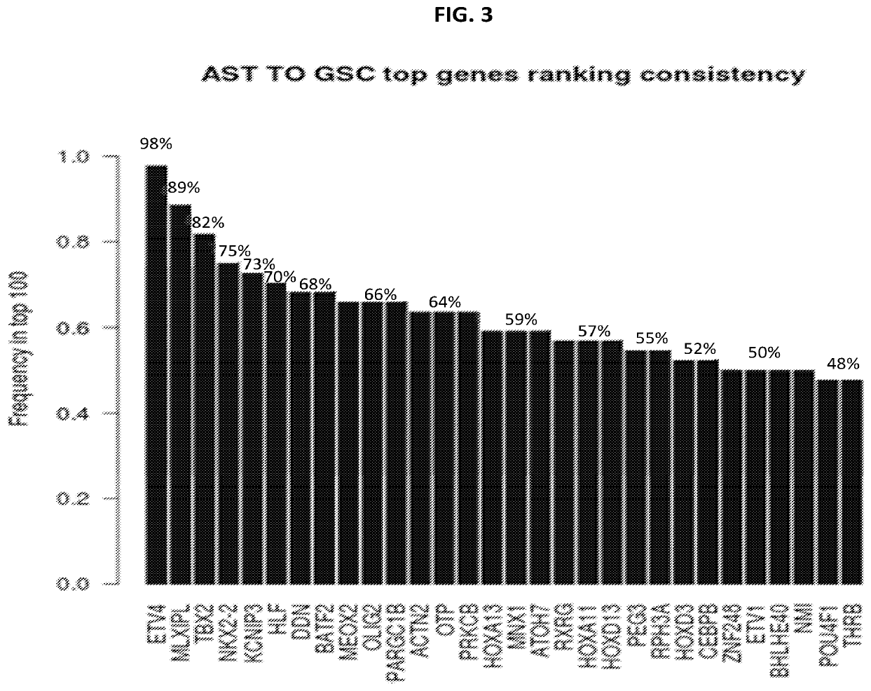

FIG. 4 illustrates the OTP methylation profile and ATAC-seq signals in GSCs and peripheral blood mononuclear cells (PBMCs). Examples of the correlation between methylation and accessibility: Methylated CpG islands=Gene not expressed=Not accessible, and vice versa.

FIG. 5 illustrates the OLIG2 methylation profile and ATAC-seq signals in GSCs and peripheral blood mononuclear cells (PBMCs). Examples of the correlation between methylation and accessibility: Methylated CpG islands=Gene not expressed=Not accessible, and vice versa.

FIG. 6 illustrates the BATF2 methylation profile and ATAC-seq signals in GSCs and peripheral blood mononuclear cells (PBMCs). Examples of the correlation between methylation and accessibility: Methylated CpG islands=Gene not expressed=Not accessible, and vice versa.

FIG. 7 illustrates the NKX2-2 methylation profile and ATAC-seq signals in GSCs and peripheral blood mononuclear cells (PBMCs). Examples of the correlation between methylation and accessibility: Methylated CpG islands=Gene not expressed=Not accessible, and vice versa.

FIG. 8 illustrates PCR products of nuclei preparations treated with transposon and transposase. Accessible regions of indicated GSC master regulator genes are disrupted by transposase specifically in GSCs but not PBMCs. The GAPDH locus was used as equal input control after transposase treatment.

FIG. 9 illustrates the results from using the gene expression approach on downstream target ID4.

FIG. 10 illustrates the results from using the gene expression approach on downstream target FREM2.

FIG. 11 illustrates the results from using the gene expression approach on downstream target NES.

FIG. 12 illustrates the results from using the gene expression approach on downstream target SALL1.

### DEFINITIONS

A “sample” comprises any tissue or material isolated from a subject, such as a patient. The sample may contain cellular and/or non-cellular material from the subject, and may contain any biological material suitable for detecting a desired biomarker, such a DNA or RNA. The sample can be isolated from any suitable biological tissue or fluid such as, but not limited to, a tissue or blood. A sample may be treated physically, chemically, and/or mechanically to disrupt tissue or cell structure, thus releasing intracellular components into a solution which may further contain enzymes, buffers, salts, detergents and the like, which are used to prepare the sample for analysis.

A “master regulator” or “cancer master regulator” is a gene or protein that acts to drive one or more intermediary gene or proteins in a pathway or network important in initiating or maintaining a cancerous state or initiating or maintaining one or more deleterious cancerous behaviors. Some master regulators are involved in pathways in the transition to a cancer state. Some master regulators are involved in pathways of aggressive (bad) cancer behavior. Expression of master regulators is indicative of poor prognosis in subjects having cancer.

A “master regulator network” refers to a master regulator and one or more genes downstream of the master regulator whose transcription level is dependent on or affected by the master regulator.

Compositions or methods “comprising” or “including” one or more recited elements may include other elements not specifically recited. For example, a composition that “comprises” or “includes” a protein may contain the protein alone or in combination with other ingredients.

Designation of a range of values includes all integers within or defining the range, and all subranges defined by integers within the range.

Unless otherwise apparent from the context, the term “about” encompasses values within a standard margin of error of measurement (e.g., SEM) of a stated value or variations±0.5%, 1%, 5%, or 10% from a specified value.

The singular forms of the articles “a,” “an,” and “the” include plural references unless the context clearly dictates otherwise. For example, the term “an antigen” or “at least one antigen” can include a plurality of antigens, including mixtures thereof.

Statistically significant means p≤0.05.

### DETAILED DESCRIPTION

Various embodiments of the inventions now will be described more fully hereinafter, in which some, but not all embodiments of the inventions are shown. Indeed, these inventions may be embodied in many different forms and should not be construed as limited to the embodiments set forth herein; rather, these embodiments are provided so that this disclosure will satisfy applicable legal requirements. The term “or” is used herein in both the alternative and conjunctive sense, unless otherwise indicated. The terms “illustrative” and “exemplary” are used to be examples with no indication of quality level.

Provided are methods for screening for cancer in a subject. The methods take advantage of the finding that cancer master regulators are often not mutated but used by cancer causative mutated genes to establish the cancer state. Many cancer master regulators are developmentally restricted and repressed epigenetically in normal adult tissues. In the cancer or pre-cancer state, the master regulator genes become unrestricted. Thus, measuring or determining the status of epigenetic markers in cancer master regulator genes can be used to detect cancer. Epigenetic markers include DNA methylation, chromosomal accessibility, and differential expression of factors downstream of a master regulator in the master regulator network.

In some embodiments, chromosomal accessibility can be measured either by disruption of a target gene locus or by insertion of exogenous DNA barcodes in the target gene locus. In some embodiments, the target gene is a master regulator. In some embodiments, master regulators of a particular cancer are identified using GeneRep/nSCORE as described in WO2018/069891, which is incorporated by reference in its entirety.

In some embodiments, ATAC-seq can be used to measure chromosomal accessibility. In some embodiments, ATAC-seq, is used to digest hypomethylated and accessible regions in DNA present in a sample. In some embodiments, ATAC-seq, is used to insert exogenous DNA barcodes into accessible regions in DNA present in a sample. The level of digestion or insertion in the area of a target region can be measured using PCR and primers designed to amplify the target region DNA. In some embodiments, the target region is a region of a master regulator, such as, but not limited to, a promoter of a master regulator.

Determining or measuring DNA accessibility may be done using methods known in the art. Exemplary methods of determining or measuring DNA accessibility include, but are not limited to, ATAC-seq, CRISPR, DNAse-seq, and MNase-seq

Determining or measuring DNA methylation may be done using methods known in the art. Exemplary methods of determining or measuring DNA methylation include, but are not limited to, ATAC-seq, digestion based assay followed by PCR, methylation specific PCR, E-ice-COLD-PCR, bead array analysis, pyrosequencing, PCR with high resolution melting, and bisulfite sequencing.

In some embodiments, ATAC-seq can be used to detect GSC in patient blood. Using ATAC-seq, accessible regions in master regulators of GCS are identified.

Described are methods assessing, detecting, monitoring the presence, or monitoring progression of cancer in a subject, or assessing or predicting prognosis or survival of a subject having cancer. The methods comprise

a) obtaining or having obtained a sample from a subject

b) measuring or having measured the chromosomal accessibility level of at least one master regulator in the sample; and

c) comparing the chromosomal accessibility level with the chromosomal accessibility level of a corresponding master regulator gene in a healthy reference sample;

wherein an increase in the chromosomal accessibility level of the at least one master regulator in the subject relative to the chromosomal accessibility level of the corresponding master regulator in the healthy reference sample indicates the possible presence of cancer in the subject, an increase or risk of increase in cancer progression in the subject, an increased risk of developing cancer in the subject, a poor prognosis, or decreased predicted survival time for the subject. The methods can be used to suggest treatments or changes in treatment of the subject. The sample can be, but is not limited to, a liquid sample. A liquid sample can be, but is not limited to, a blood sample.

Described are methods assessing, detecting, monitoring the presence, or monitoring progression of cancer in a subject, or assessing or predicting prognosis or survival of a subject having cancer. The methods comprise

a) obtaining or having obtained a sample from a subject

b) measuring or having measured the chromosomal DNA methylation level of at least one master regulator in the sample; and

c) comparing the chromosomal DNA methylation level with the chromosomal DNA methylation level of a corresponding master regulator gene in a healthy reference sample;

wherein a decrease in the chromosomal DNA methylation level of the at least one master regulator in the subject relative to the chromosomal DNA methylation level of the corresponding master regulator in the healthy reference sample indicates the possible presence of cancer in the subject, an increase or risk of increase in cancer progression in the subject, an increased risk of developing cancer in the subject, a poor prognosis, or decreased predicted survival time for the subject. The methods can be used to suggest treatments or changes in treatment of the subject. The sample can be, but is not limited to, a liquid sample. A liquid sample can be, but is not limited to, a blood sample. In some embodiments, chromosomal DNA methylation is measure in a promoter or regulator region of at least one master regulator gene.

Described are methods assessing, detecting, monitoring the presence, or monitoring progression of cancer in a subject, or assessing or predicting prognosis or survival of a subject having cancer. The methods comprise

a) obtaining or having obtained a sample from a subject

b) measuring or having measured the expression level of at least gene downstream of a master regulator in the master regulator network in the sample (downstream gene); and

c) comparing the expression level with the expression level of a corresponding downstream gene in a healthy reference sample;

wherein an increase or decrease in expression level of the at least one downstream gene in the subject relative to the expression level of the corresponding gene in the healthy reference sample indicates the possible presence of cancer in the subject, an increase or risk of increase in cancer progression in the subject, an increased risk of developing cancer in the subject, a poor prognosis, or decreased predicted survival time for the subject. The methods can be used to suggest treatments or changes in treatment of the subject. The sample can be, but is not limited to, a liquid sample. A liquid sample can be, but is not limited to, a blood sample.

Methods of determining gene expression in a sample can be performed using methods known in the art. Such methods included, but are not limited to, nucleotide amplification assays (including but not limited to PCR, RT-PCR, serial analysis of gene expression, and differential display), microarray technologies, proteomics, HPLC, and Western electrophoresis.

In some embodiments, the methods are used to assess whether a subject has a decreased predicted survival time for cancer comprising: determining a level of chromosomal accessibility or chromosomal DNA methylation of a cancer master regulator in a sample from the subject, wherein an increase chromosomal accessibility level of the at least one master regulator relative to the chromosomal accessibility level of the corresponding master regulator in a healthy reference sample, or a decrease chromosomal DNA methylation level of the at least one master regulator relative to the chromosomal DNA methylation level of the corresponding master regulator in a healthy reference sample is indicative that the subject has a poor survival prognosis for the cancer.

In some embodiments, chromosomal accessibility or chromosomal DNA methylation levels of 2, 3, 4, 5, 6, 7, 8, 9, 10. 11, 12, 13, 14, 15, 16, 17, 18, 19, or 20 master regulators in a subject sample are measured and compared with the chromosomal accessibility or chromosomal DNA methylation level of the corresponding master regulators in a healthy reference (control) sample.

In some embodiments, the methods are used to assess whether a subject has a decreased predicted survival time for cancer comprising: determining an expression level of a least one gene downstream of a cancer master regulator in the cancer master regulator network (downstream gene) in a sample from the subject, wherein an change in expression level of the at least one downstream gene relative to the expression level of the corresponding gene in a healthy reference sample is indicative that the subject has a poor survival prognosis for the cancer.

In some embodiments, a change in expression levels of 2, 3, 4, 5, 6, 7, 8, 9, 10. 11, 12, 13, 14, 15, 16, 17, 18, 19, or 20 downstream gene in a subject sample are measured and compared with the expression levels of the corresponding downstream genes in a healthy reference (control) sample.

In some embodiments, glioblastoma is detected in a patient by analyzing differential expression of 1, 2, 3, or 4 of ID4, FREM2, NES, and SALL1 in a blood sample.

In some embodiments, chromosomal accessibility for one or more master regulators and chromosomal DNA methylation for one or more master regulators is measured. The master regulators can be for the same cancer type. Chromosomal accessibility and chromosomal DNA methylation can be measured for the same master regulators, different master regulator or overlapping sets of master regulators.

In some embodiments, measurement of chromosomal accessibility and/or chromosomal DNA methylation for one or more master regulators is combined with measurement of differential expression of one or more downstream genes.

In some embodiments, master regulators are selected based on the cancer type.

Expression of master regulator genes in cancer drive bad cancer behavior or poor prognosis of the cancer. Poor prognosis can include, but is not limited to, poor response to typical cancer treatment, aggressive cancer growth, increased metastasis, and/or decreased survival time. Identification of poor prognosis in a patient can be used to diagnose and/or prescribe treatment. Such treatment can include, but is not limited to, master regulator-specific treatment and/or more aggressive treatment. Master regulator-specific treatment includes treatments, including adjuvants, known to be effective in treating similar cancers in other patients expressing the same master regulator gene(s). As an example, patients having increased expression of VDR or VDR-related genes may be given vitamin D.

### EXAMPLES

### Example 1: Cancer Screening Method: Chromosomal Accessibility Approach

The regulatory chromosomal elements of master regulators of GSCs, especially those that are developmentally restricted, are accessible in GSCs and not accessible in adult normal cells. Developmentally restricted genes are highly methylated and thus highly coiled and folded in normal adult cells and are therefore not accessible to transcription factors so that they are not expressed haphazardly. In contrast, these master regulators in cancer cells are unmethylated and therefore accessible. Additionally, cancer stem cells tend to leave the primary tumor and spread which leads to metastases. Therefore, migrating GSCs can be detected by measuring and amplifying the accessibility of these regulatory chromosomal elements among other normal adult cells, e.g., blood.

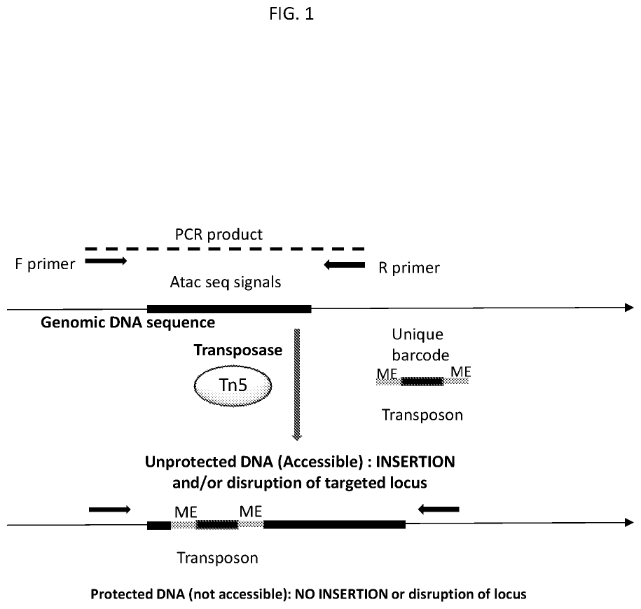

Methods such as ATAC-seq (FIG. 1) or CRISPR in combination with qPCR or nanostring/multiplexing can be used to access and detect the unmethylated DNA of the master regulators of cancer in the blood of a subject. Additionally, technologies that can access DNA, cleave DNA, and insert a barcode can also be used.

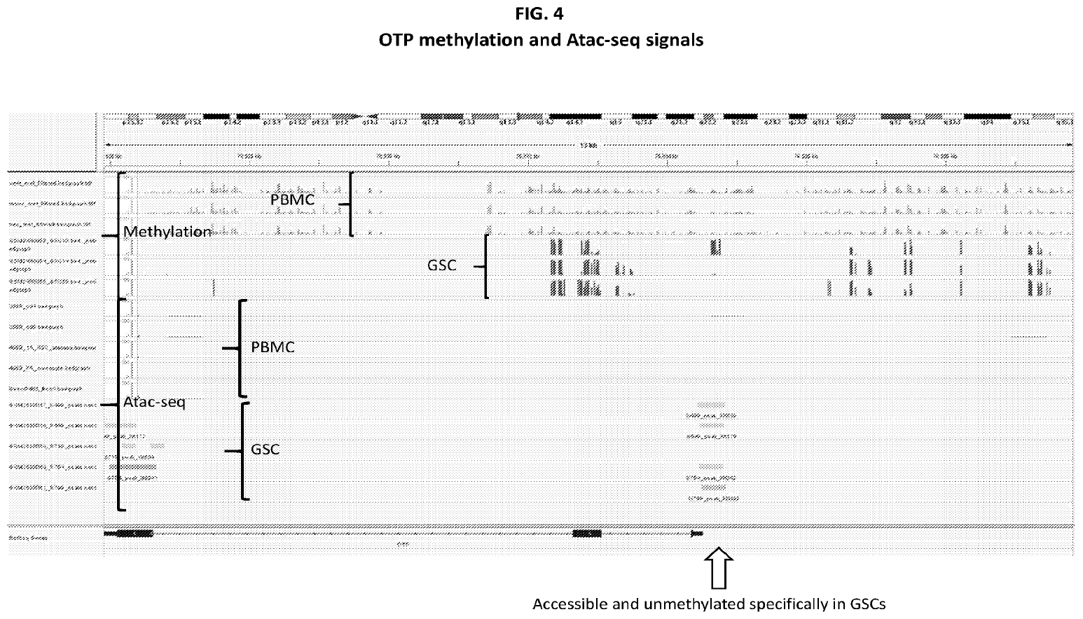

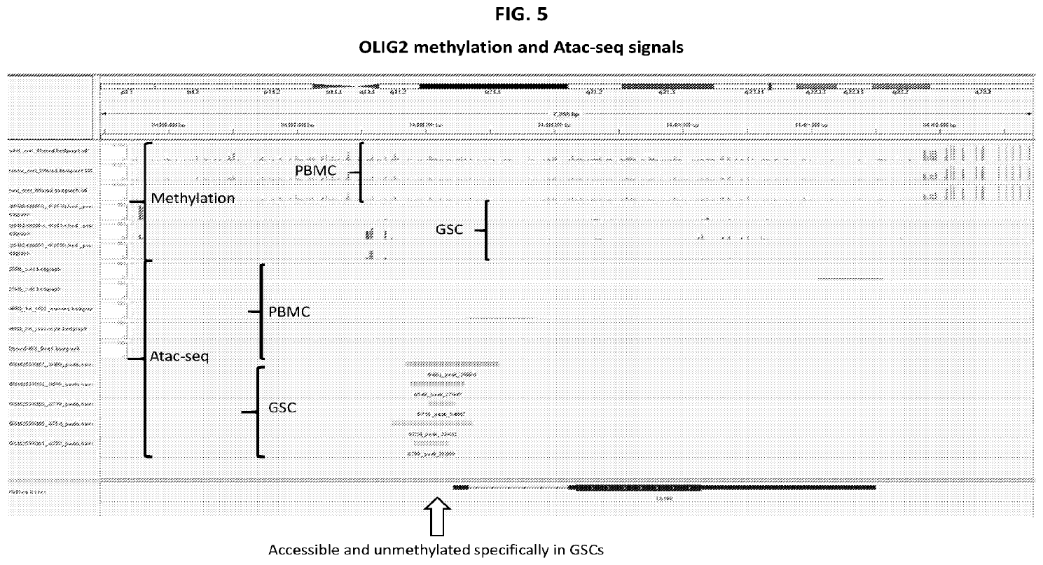

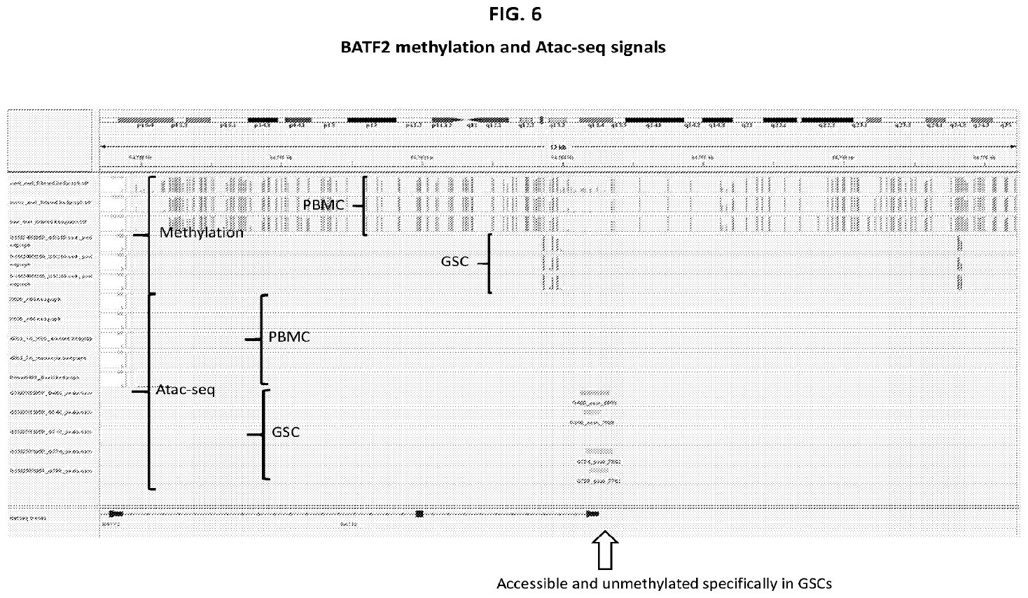

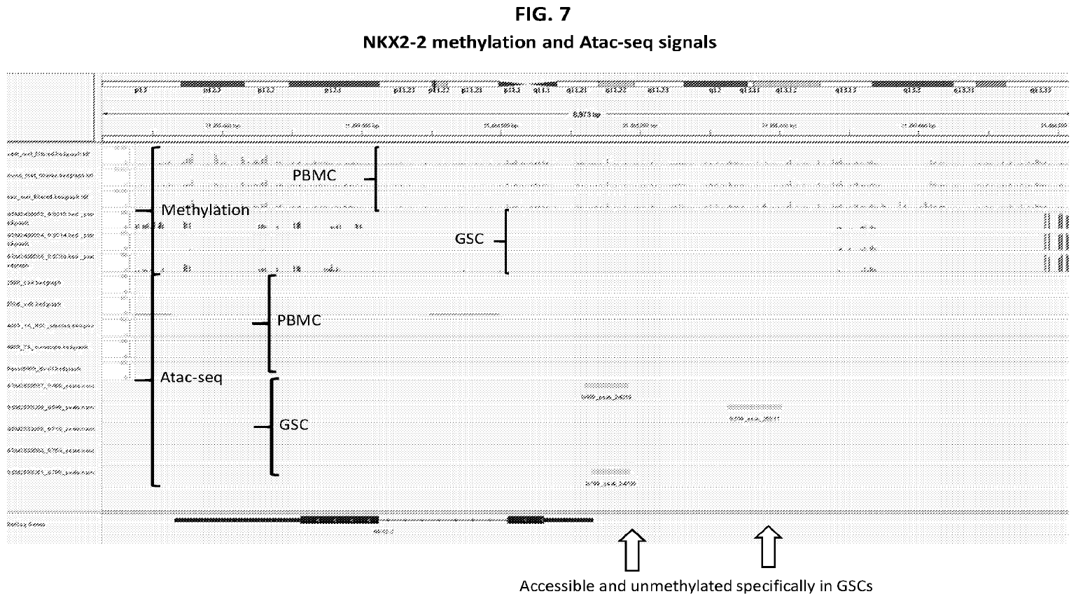

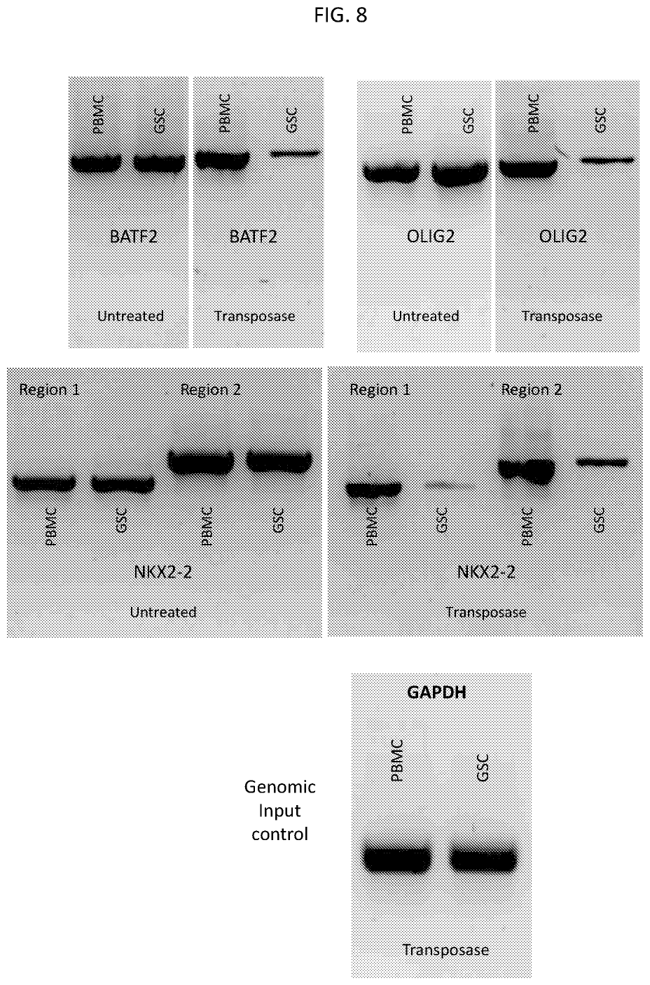

FIGS. 4-7 show methylation profiles and ATAC-seq signals in GSCs and four master regulators of GSCs, OTP, OLIG2, BATF2, and NKX2-2. FIG. 8 shows PCR products of nuclei preparations treated with transposon and transposase and shows that accessible regions of indicated GSC master regulator genes are disrupted by transposase specifically in GSCs but not PBMCs. Thus, ATAT-seq mediated degradation of, disruption of, or insertions into OTP, OLIG2, BATF2, and/or NKX2-2 can be used in the diagnosis of GSC. Further, the ATAC-seq assay can be used to detect and diagnose GCS using blood samples.

The same approach can be used to detect cancer stem cells from other cancers by identifying the master regulators of cancer.

### Example 2. Cancer Screening Method: Chromosomal Accessibility Approach

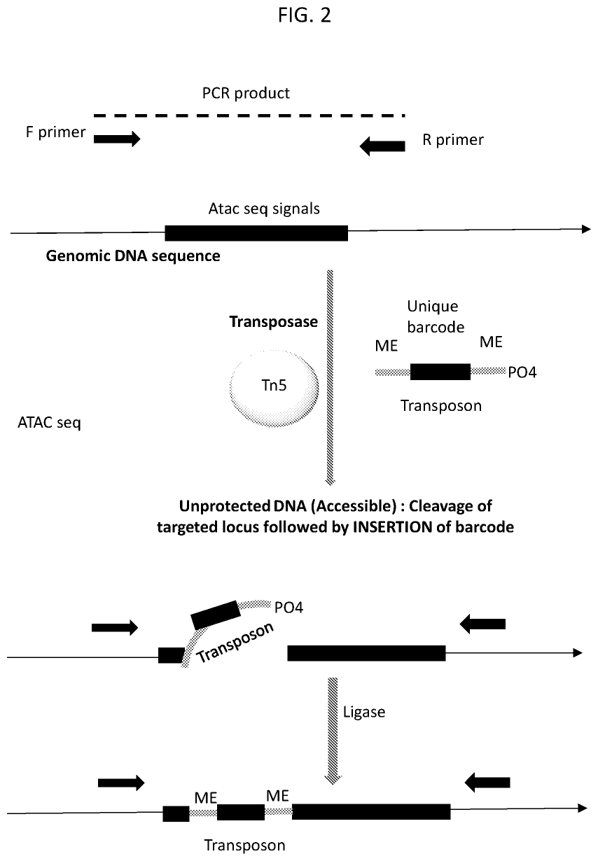

The data in example 1 was generated using a transposon without a phosphate (PO₄) at the end of the transposon. In the absence of the terminal PO₄ (FIG. 1) the transposase can destroy the target promoter loci since ligation can't proceed. Addition of the PO₄ at the end of the transposon (FIG. 2) permits testing of the insertion of the unique transposon specifically at the accessible regions in master regulator gene promoters. This results in a larger amplified product (either as a ladder or a smear) extending upward from the native product, using the primer F and R pair.

### Example 3. Master Regulators

Using genetic analyses, such as GeneRep/nSCORE, the top 5-20 genes in the GSC regulatory network with the highest differential expression between GSC and PBMC are identified. Analysis of master regulators is then combined with downstream factors, such as OTP, OLIG2, BATF2, and NKX2-2, to detect both upstream master regulators and downstream pathways at the same time to simultaneously increase sensitivity and specificity for the screening technology.

### Example 4: Cancer Screening Method: Gene Expression Approach

Master regulators are expressed at a low level and therefore directly measuring the expression level of the master regulators in the blood can be unreliable. However, target genes (and their expression protein products) downstream of GSC master regulators amplify GBM state signals to maintain the GBM state. The expression of these downstream factors can reach hundreds of fold higher than in normal healthy adult cells and can be used to detect rare migratory GSCs, e.g., in blood. The same approach can be used to detect cancer stem cells from other cancers by identifying the downstream genes.

We identified the following downstream factors with the highest levels of expression in GSCs compared to PBMCs: ENSG00000277459, SALL1, ID4, FOXG1, FJX1, FREM2, NES, TUBB2B, MSI1, FKBP10, CBARP, GNG12, CD276, FAT1, CTXN1, DPYSL5, TYRO3, GJC1, CHST3, LRP4, PHF21B, ADAMTS9, CSPG5, TEAD1, BCAR1, EML1, RBFOX2, MPDZ, MSX1, EFNB3, ENAH, LYPD6, GTF2IRD1, TBX2, ANK2, C1QL1, ZIC1, EMP2, TMEM132A, CX3CL1, SYDE1, SLC16A2, SOX9, RND3, LARP6, CNN3, SPRY4, DPF1, PCGF2, BOC, OBSL1, SOX2, EPB41L1, MEX3A, NCS1, SMO, TMC7, SEZ6L2, POU3F2, FAM171A2, DENND2A, TANC1, PROX1, ENSG00000268592, PNPLA3, TSKU, DLX1, KIAA1549, MTSS1L, PTPRF, IRX5, DZIP1, MAGI1, ADCY6, BCHE, CXADR, TEAD4, PTPN21, CDR2L, ADGRL3, REEP2, SHROOM3, ARC, EEF1A2, ETV4, EGFR, UCHL1, KAZALD1, TJP1, ENSG00000237004, ETV5, CKB, KCNF1, MAP1B, S100A16, COL27A1, VGF, ALDH7A1, GAS1, LOC101927480, CITED1, ETV1, NOVA1, JPH1, FBXO17, CNKSR3, PDXP, PLEKHH3, PYGO1, SCARA3, RTL8B, LAMB1, MYH14, CASKIN1, NLGN2, PACSIN3, CA12, ARHGAP39, LAMA4, ZNF462, NR2F1, CSRP2, ARHGEF25, GLI3, TMEFF1, B4GALNT1, SH3D19, SV2A, VAX2, CASC10, CSPG4, SNAI2, ARSJ, FSCN1, KHDRBS3, RASSF8, SMARCA1, TNC, PIR, ANTXR1, PHLDB1, CASKIN2, LAMC1, PAX6, ASPHD1, MAPK12, CAVIN1, TSPAN6, SEMA6D, NDRG4, PRR36, PFN2, SOX21, SPTBN2, GPC1, NRSN2, AADAT, GNA11, TCEAL9, DDAH1, KIAA1549L, LGR4, MEX3D, TNKS1BP1, PIMREG, SCD, FRMD6, HUNK, TMEM136, LRRC49, ARNT2, ENSG00000259495, DOCK1, PTPRS, TTC23, PPFIBP1, LHX2, SIX4, MAPK8IP1, IGDCC3, DMRTA2, STXBP1, PTPN14, SLC2A10, ARMC9, TBC1D16, FLNC, RHPN2, RHBDF1, P3H4, ENSG00000261578, DTNA, CELSR2, NOVA2, GPR176, VPS37D, SLC26A10, DNAJC22, ZNRF3. Using a series of these factors, one can build a biomarker structure highly specific to GSCs compared to blood cells.

TABLE 1
Downstream factors with higher levels of expression in
GSCs compared to PBMCs.

| Gene | min_GSC_vs_max_blood |
|---|---|
| ENSG00000277459 | 832.3084162 |
| SALL1 | 458.4426045 |
| ID4 | 337.4452973 |
| FREM2 | 161.4199241 |
| NES | 155.6845922 |
| TUBB2B | 149.6286058 |
| MSI1 | 145.1664194 |

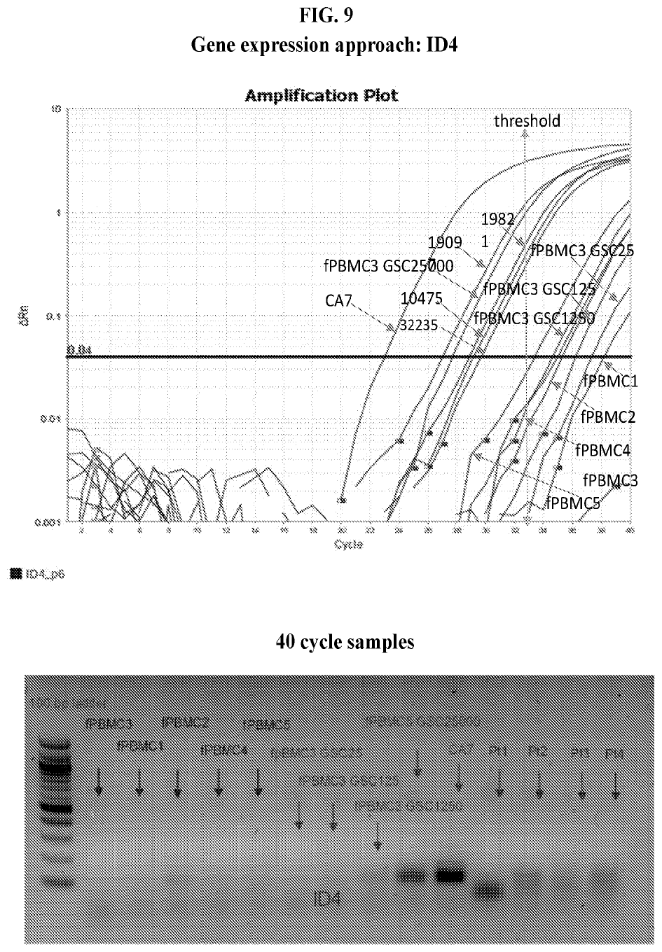

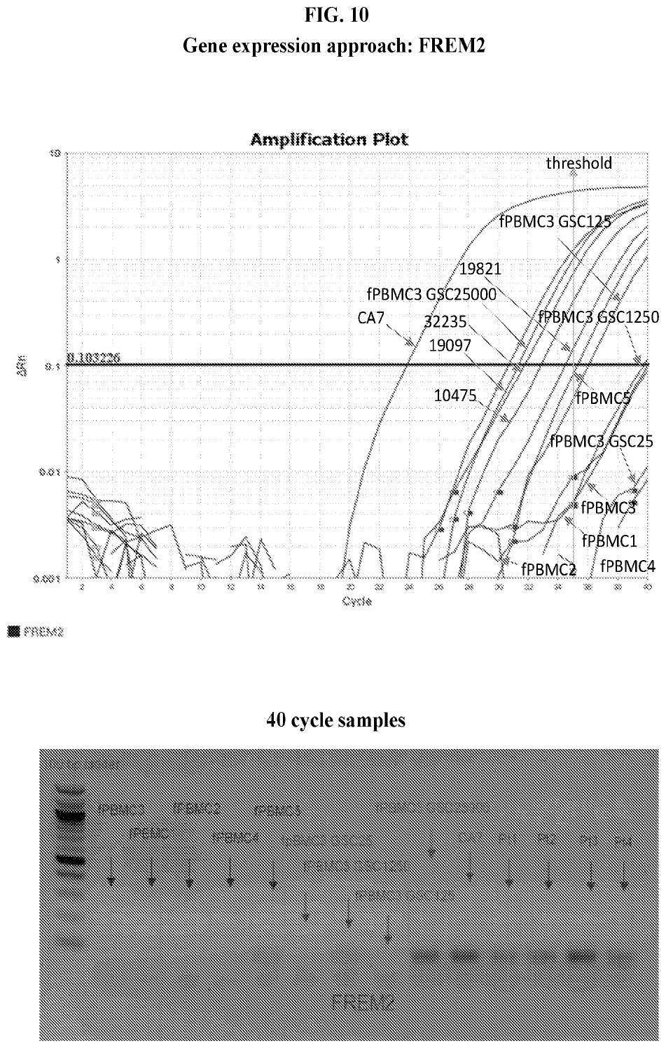

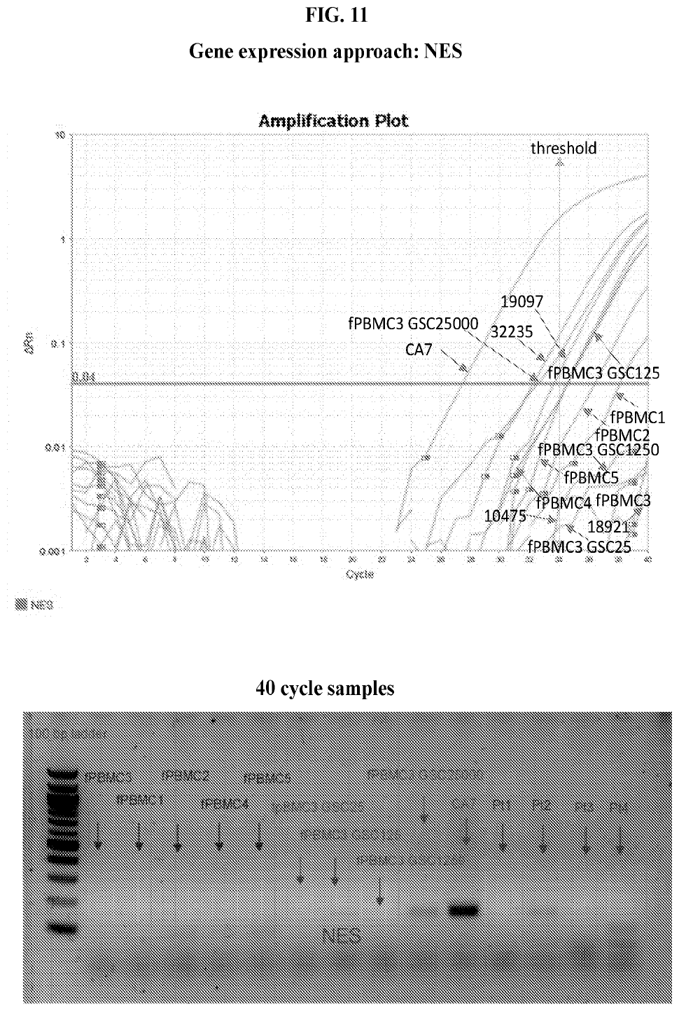

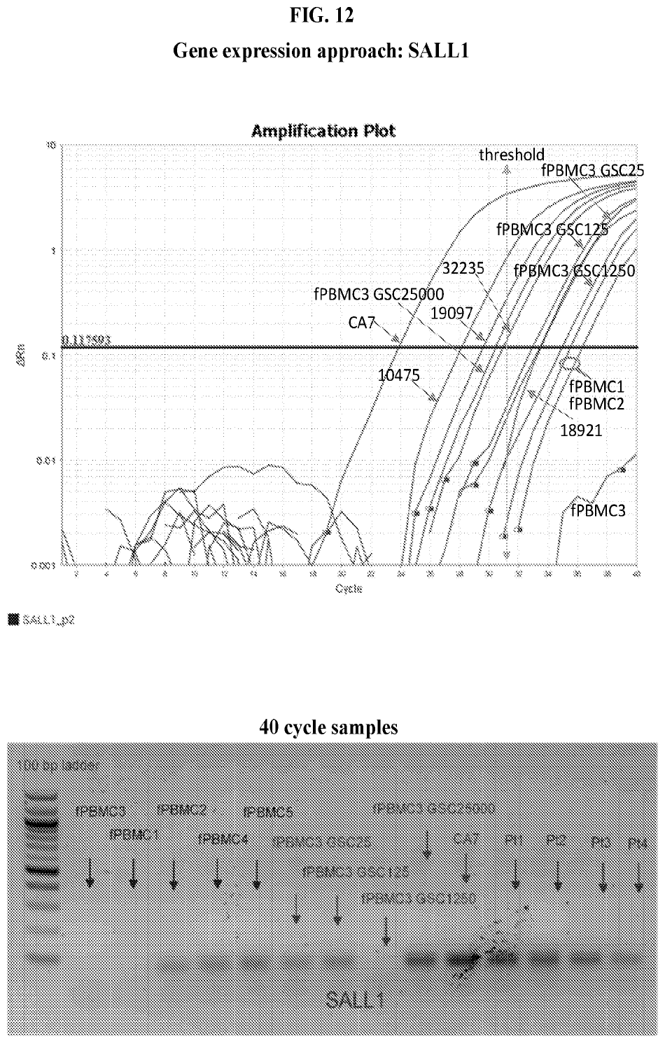

For this study we used PCR to look at gene expression of four of the downstream factors: ID4, FREM2, NES, and SALL1 (FIGS. 9-12, Tables 2-9). The CT (cycle threshold) is defined as the number of cycles required for the fluorescent signal to cross the threshold, i.e., exceed the background level. CT levels are inversely proportional to the amount of target nucleic acid in the sample, i.e., the lower the CT level the greater the amount of target nucleic acid in the sample. Here, we have shown that expression of downstream factors ID4, FREM2, NES, and SALL1 is higher in GBM patient samples than in control healthy patient samples (FIGS. 9-12). The blue panel in FIGS. 9-12 shows results from control healthy patient blood from Red Cross, and each sample represents a pooled sample of three donors. The yellow/orange panels show results from an experiment testing the detection threshold for the qPCR method used where control samples are spiked with GSCs. The pink panels show results from four GBM patients. It is therefore possible to measure expression of master regulators indirectly by measuring expression of downstream factors in patient blood.

TABLE 2
PCR analysis of ID4.

| Sample | Target | | | |
|---|---|---|---|---|
| Name | Name | CT | Ct Mean | ACTB |
| fPBMC3 | ID4 | Undetermined | Undetermined | 21.80076 |
| fPBMC3 | ID4 | Undetermined | Undetermined | |
| fPBMC3 | ID4 | Undetermined | Undetermined | |
| fPBMC1 | ID4 | 38.158 | 36.485 | 16.243 |
| fPBMC1 | ID4 | 35.667 | 36.485 | |
| fPBMC1 | ID4 | 35.629 | 36.485 | |
| fPBMC2 | ID4 | 35.408 | 34.231 | 15.557 |
| fPBMC2 | ID4 | 34.347 | 34.231 | |
| fPBMC2 | ID4 | 32.939 | 34.231 | |
| fPBMC4 | ID4 | 34.974 | 35.172 | 16.288 |
| fPBMC4 | ID4 | 34.728 | 35.172 | |
| fPBMC4 | ID4 | 35.816 | 35.172 | |
| fPBMC5 | ID4 | 33.462 | 33.686 | 16.989 |
| fPBMC5 | ID4 | 33.689 | 33.686 | |
| fPBMC5 | ID4 | 33.906 | 33.686 | |

TABLE 3
PCR analysis of ID4.

| Sample | Target | | Ct | |
|---|---|---|---|---|
| Name | Name | CT | Mean | ACTB |
| fPBMC3 GSC25 | ID4 | 37.307 | 37.380 | 21.163 |
| fPBMC3 GSC25 | ID4 | 37.454 | 37.380 | |
| fPBMC3 GSC25 | ID4 | Undetermined | 37.380 | |
| fPBMC3 GSC125 | ID4 | 36.327 | 35.782 | 19.422 |
| fPBMC3 GSC125 | ID4 | 35.864 | 35.782 | |
| fPBMC3 GSC125 | ID4 | 35.155 | 35.782 | |
| fPBMC3 GSC1250 | ID4 | Undetermined | 33.750 | 19.744 |
| fPBMC3 GSC1250 | ID4 | 34.705 | 33.750 | |
| fPBMC3 GSC1250 | ID4 | 32.795 | 33.750 | |
| fPBMC3 GSC25000 | ID4 | 27.774 | 28.606 | 19.380 |
| fPBMC3 GSC25000 | ID4 | 28.845 | 28.606 | |
| fPBMC3 GSC25000 | ID4 | 29.200 | 28.606 | |
| CA7 2000 | ID4 | 23.076 | 22.892 | 17.411 |
| CA7 2000 | ID4 | 22.783 | 22.892 | |
| CA7 2000 | ID4 | 22.816 | 22.892 | |
| Pt1 | | | | |
| 10475 | ID4 | 29.354 | 29.007 | 17.601 |
| 10475 | ID4 | 28.367 | 29.007 | |
| 10475 | ID4 | 29.301 | 29.007 | |
| Pt2 | | | | |
| 32235 | ID4 | 29.774 | 31.233 | 14.911 |
| 32235 | ID4 | 32.338 | 31.233 | |
| 32235 | ID4 | 31.588 | 31.233 | |
| Pt3 | | | | |
| 19907 | ID4 | 27.033 | 27.599 | 19.393 |
| 19907 | ID4 | 27.813 | 27.599 | |
| 19907 | ID4 | 27.952 | 27.599 | |
| Pt4 | | | | |
| 18921 | ID4 | 28.937 | 28.987 | 15.936 |
| 18921 | ID4 | 28.776 | 28.987 | |
| 18921 | ID4 | 29.247 | 28.987 | |

TABLE 4
PCR analysis of FREM2.

| Sample | Target | | | |
|---|---|---|---|---|
| Name | Name | CT | Ct Mean | ACTB |
| fPBMC3 | FREM2 | Undetermined | Undetermined | 22.451 |
| fPBMC3 | FREM2 | Undetermined | Undetermined | |
| fPBMC3 | FREM2 | Undetermined | Undetermined | |
| fPBMC1 | FREM2 | Undetermined | Undetermined | 17.402 |
| fPBMC1 | FREM2 | Undetermined | Undetermined | |
| fPBMC2 | FREM2 | Undetermined | Undetermined | 16.042 |
| fPBMC2 | FREM2 | Undetermined | Undetermined | |
| fPBMC4 | FREM2 | Undetermined | Undetermined | 17.197 |
| fPBMC4 | FREM2 | Undetermined | Undetermined | |
| fPBMC5 | FREM2 | 35.393 | 35.914 | 17.821 |
| fPBMC5 | FREM2 | 36.434 | 35.914 | |

TABLE 5
PCR analysis of FREM2.

| | Target | | | |
|---|---|---|---|---|
| Sample Name | Name | CT | Ct Mean | ACTB |
| fPBMC3 GSC25 | FREM2 | Undetermined | Undetermined | 22.354 |
| fPBMC3 GSC25 | FREM2 | Undetermined | Undetermined | |
| fPBMC3 GSC25 | FREM2 | Undetermined | Undetermined | |
| fPBMC3 GSC125 | FREM2 | 36.170 | 36.296 | 20.173 |
| fPBMC3 GSC125 | FREM2 | 38.124 | 36.296 | |
| fPBMC3 GSC125 | FREM2 | 34.593 | 36.296 | |
| fPBMC3 GSC1250 | FREM2 | Undetermined | 39.730 | 21.953 |
| fPBMC3 GSC1250 | FREM2 | Undetermined | 39.730 | |
| fPBMC3 GSC1250 | FREM2 | 39.730 | 39.730 | |
| fPBMC3 GSC25000 | FREM2 | 30.768 | 30.699 | 19.789 |
| fPBMC3 GSC25000 | FREM2 | 31.001 | 30.699 | |
| fPBMC3 GSC25000 | FREM2 | 30.328 | 30.699 | |
| CA7 2000 | FREM2 | 23.897 | 24.108 | 18.294 |
| CA7 2000 | FREM2 | 23.915 | 24.108 | |
| CA7 2000 | FREM2 | 24.510 | 24.108 | |
| Pt1 | | | | |
| 10475 | FREM2 | 34.720 | 34.095 | 18.272 |
| 10475 | FREM2 | 32.916 | 34.095 | |
| 10475 | FREM2 | 34.650 | 34.095 | |
| Pt2 | | | | |
| 32235 | FREM2 | 31.853 | 32.483 | 15.748 |
| 32235 | FREM2 | 32.611 | 32.483 | |
| 32235 | FREM2 | 32.986 | 32.483 | |
| Pt3 | | | | |
| 19097 | FREM2 | 31.443 | 31.167 | 19.934 |
| 19097 | FREM2 | 30.647 | 31.167 | |
| 19097 | FREM2 | 31.412 | 31.167 | |
| Pt4 | | | | |
| 18921 | FREM2 | 34.565 | 33.675 | 16.627 |
| 18921 | FREM2 | 33.358 | 33.675 | |
| 18921 | FREM2 | 33.100 | 33.675 | |

TABLE 6
PCR analysis of NES.

| Sample | Target | | | |
|---|---|---|---|---|
| Name | Name | CT | Ct Mean | ACTB |
| fPBMC3 | NES | Undetermined | Undetermined | 22.451 |
| fPBMC3 | NES | Undetermined | Undetermined | |
| fPBMC3 | NES | Undetermined | Undetermined | |
| fPBMC1 | NES | 38.107 | 38.107 | 17.402 |
| fPBMC1 | NES | Undetermined | 38.107 | |
| fPBMC2 | NES | 36.521 | 36.223 | 16.042 |
| fPBMC2 | NES | 35.926 | 36.223 | |
| fPBMC4 | NES | 34.633 | 36.677 | 17.197 |
| fPBMC4 | NES | 38.721 | 36.677 | |
| fPBMC5 | NES | Undetermined | 34.639 | 17.821 |
| fPBMC5 | NES | 34.639 | 34.639 | |

TABLE 7
PCR analysis of NES.

| | Target | | | |
|---|---|---|---|---|
| Sample Name | Name | CT | Ct Mean | ACTB |
| fPBMC3 GSC25 | NES | Undetermined | Undetermined | 22.354 |
| fPBMC3 GSC25 | NES | Undetermined | Undetermined | |
| fPBMC3 GSC25 | NES | Undetermined | Undetermined | |
| fPBMC3 GSC125 | NES | Undetermined | 34.619 | 20.173 |
| fPBMC3 GSC125 | NES | 34.619 | 34.619 | |
| fPBMC3 GSC125 | NES | Undetermined | 34.619 | |
| fPBMC3 GSC1250 | NES | Undetermined | Undetermined | 21.953 |
| fPBMC3 GSC1250 | NES | Undetermined | Undetermined | |
| fPBMC3 GSC1250 | NES | Undetermined | Undetermined | |
| fPBMC3 GSC25000 | NES | 33.184 | 33.567 | 19.789 |
| fPBMC3 GSC25000 | NES | 32.671 | 33.567 | |
| fPBMC3 GSC25000 | NES | 34.845 | 33.567 | |
| CA7 2000 | NES | 27.880 | 27.279 | 18.294 |
| CA7 2000 | NES | 27.529 | 27.279 | |
| CA7 2000 | NES | 26.428 | 27.279 | |
| Pt1 | | | | |
| 10475 | NES | Undetermined | Undetermined | 18.272 |
| 10475 | NES | Undetermined | Undetermined | |
| 10475 | NES | Undetermined | Undetermined | |
| Pt2 | | | | |
| 32235 | NES | 32.337 | 32.466 | 15.748 |
| 32235 | NES | 32.595 | 32.466 | |
| 32235 | NES | Undetermined | 32.466 | |
| Pt3 | | | | |
| 19097 | NES | 33.737 | 37.104 | 19.934 |
| 19097 | NES | 38.399 | 37.104 | |
| 19097 | NES | 39.175 | 37.104 | |
| Pt4 | | | | |
| 18921 | NES | Undetermined | 33.572 | 16.627 |
| 18921 | NES | 34.457 | 33.572 | |
| 18921 | NES | 32.686 | 33.572 | |

TABLE 8
PCR analysis of SALL1.

| Sample | Target | | | |
|---|---|---|---|---|
| Name | Name | CT | Ct Mean | ACTB |
| fPBMC3 | SALL1 | Undetermined | Undetermined | 16.920 |
| fPBMC3 | SALL1 | Undetermined | Undetermined | |
| fPBMC1 | SALL1 | Undetermined | 36.385 | 16.920 |
| fPBMC1 | SALL1 | 36.385 | 36.385 | |
| fPBMC1 | SALL1 | Undetermined | 36.385 | |
| fPBMC2 | SALL1 | 34.407 | 35.259 | 15.757 |
| fPBMC2 | SALL1 | 35.803 | 35.259 | |
| fPBMC2 | SALL1 | 35.566 | 35.259 | |
| fPBMC4 | SALL1 | 34.385 | 33.410 | 16.288 |
| fPBMC4 | SALL1 | 32.931 | 33.410 | |
| fPBMC4 | SALL1 | 32.915 | 33.410 | |
| fPBMC5 | SALL1 | 30.987 | 31.534 | 16.989 |
| fPBMC5 | SALL1 | 31.947 | 31.534 | |
| fPBMC5 | SALL1 | 31.668 | 31.534 | |

TABLE 9
PCR analysis of SALL1.

| | Target | | | |
|---|---|---|---|---|
| Sample Name | Name | CT | Ct Mean | ACTB |
| fPBMC3 GSC25 | SALL1 | 34.921 | 34.204 | 22.287 |
| fPBMC3 GSC25 | SALL1 | 33.487 | 34.204 | |
| fPBMC3 GSC125 | SALL1 | 33.680 | 33.331 | 19.857 |
| fPBMC3 GSC125 | SALL1 | 32.982 | 33.331 | |
| fPBMC3 GSC1250 | SALL1 | Undetermined | 34.954 | 21.335 |
| fPBMC3 GSC1250 | SALL1 | 34.954 | 34.954 | |
| fPBMC3 GSC25000 | SALL1 | 30.514 | 30.508 | 19.993 |
| fPBMC3 GSC25000 | SALL1 | 30.502 | 30.508 | |
| CA7 9000 | SALL1 | 24.335 | 24.114 | 18.154 |
| CA7 9000 | SALL1 | 23.894 | 24.114 | |
| Pt1 | | | | |
| 10475 | SALL1 | 28.261 | 28.139 | 18.058 |
| 10475 | SALL1 | 28.210 | 28.139 | |
| 10475 | SALL1 | 27.946 | 28.139 | |
| Pt2 | | | | |
| 32235 | SALL1 | 30.978 | 31.177 | 15.398 |
| 32235 | SALL1 | 30.973 | 31.177 | |
| 32235 | SALL1 | 31.580 | 31.177 | |
| Pt3 | | | | |
| 19097 | SALL1 | 30.222 | 29.933 | 19.509 |
| 19097 | SALL1 | 29.643 | 29.933 | |
| Pt4 | | | | |
| 18921 | SALL1 | 33.406 | 33.460 | 16.743 |
| 18921 | SALL1 | 33.515 | 33.460 | |

The same concept can be used for other cancers in which expression profiles of their cancer stem cells are available.

### Example 5. PCR Detection Optimization

We showed that using qPCR there is a limit of sensitivity due to the rarity of circulating GBM cells in the blood. In the above example, 5 GBM per 1 million blood cells was reliably detected. Reliable detection at a sensitivity of 1 GBM cell per 1 million of blood cells to 1 GBM cell for 5 million blood cells is desired.

A. Blood cell depletion: In order to enhance sensitivity, blood cells were depleted from the samples to enrich for cancer cells. We used a magnetic method to deplete CD45+ cells or immune cells (FIG. 1B) right). To test the method, normal peripheral blood mononuclear cells (PBMC) were spiked with GBM stem cells (GSC) at different frequencies, CD45+ cells were depleted, and sensitivity of detection was analyzed. For these tests, the FREM2 target was used.

Without enrichment, a frequency of 5 cancer cells per 1 million blood cells (PBMC+GSC 5) was the lowest reliable limit of detection. Surprisingly, depletion of CD545+ cells did not improve sensitivity. On the contrary, depletion of CD45+ cells, decreased sensitivity of FREM2 detection. One likely explanation was that cancer cells bound to the magnetic beads non-specifically, thereby removing them from the sample with the CD45+ cells (FIG. 2B).

TABLE 10
Input 50 ng RNA/well

| Sample Name | Target Name | CT | GAPDH |
|---|---|---|---|
| L2 | FREM2 | 25.961 | 30.648 |
| PBMC + GSC 100 | FREM2 | 32.844 | 31.512 |
| PBMC + GSC 50 | FREM2 | 35.426 | 32.926 |
| PBMC + GSC 5 | FREM2 | 38.964 | 32.623 |
| PBMC + GSC 1 | FREM2 | undetermined | 32.651 |
| PBMC | FREM2 | 35.543 | 38.596 |

B. RNA level: We next tested increasing the RNA input in the no enrichment sample. With a high input level, non-specific amplification became a problem. With 100 ng of RNA per well we could no longer detect less than 50 cancer cells per 1 million blood cells. Non-specific binding of the PCR primers to the excess RNA template limited the level of detection.

TABLE 11
Input 100 ng/well

| Sample Name | Target Name | CT | GAPDH |
|---|---|---|---|
| L2 | FREM2 | 28.079 | 32.931 |
| PBMC + GSC 100 | FREM2 | 34.308 | 33.524 |
| PBMC + GSC 50 | FREM2 | 36.965 | 34.697 |
| PBMC + GSC 5 | FREM2 | 38.321 | 33.708 |
| PBMC + GSC 1 | FREM2 | 38.615 | 31.675 |
| PBMC | FREM2 | 38.746 | 32.918 |

C. Two-step headstart PCR: 10 cycles of PCR were used to amplify and molecularly enrich rare RNA species. The amplification product of this first round of PCR was then used as input for the next 30 cycles of PCR amplification with the same primer pair. Two-step headstart PCR, increased sensitivity top reliably detect 1 cancer cell per 1 million blood cells (FIG. 4B).

TABLE 12
Two-step headstart PCR, input 50 ng RNA /well.

| Sample Name | Target Name | CT | GAPDH |
|---|---|---|---|
| L2 | FREM2 | 17.941 | 23.159 |
| PBMC + GSC 100 | FREM2 | 23.738 | 24.006 |
| PBMC + GSC 50 | FREM2 | 26.867 | 24.794 |
| PBMC + GSC 5 | FREM2 | 28.191 | 22.680 |
| PBMC + GSC 1 | FREM2 | 28.984 | 24.356 |
| PBMC | FREM2 | undetermined | 23.260 |

D. Nested two-step headstart PCR. A non-enriched sample is PCR amplified for 10 cycles with a first primer pair, primer pair A. The product from this first round of PCR is then used as input for 30-40 more cycles in a second round of PCR amplification using a second, nested primer pair, primer pair B. Primer pair B amplifies a region of DNA contained within the primer pair A amplification product. In other embodiments, the first PCR reaction is 5-15 cycles and the second PCR reaction is 20-40 cycles.

Using nested two-step headstart PCR and 1-5 tumor cells per 1 million blood cells, reliable detection of cancer cells was observed. The data in Table 13 show that the nested method separated the lower end frequencies, 1 tumor cell vs 5 tumor cells, with CT difference of almost 4. Thus, nested two-step headstart PCR was able to detect low tumor cell frequencies. Based on these results, it is expected that nested two-step headstart PCR will work for detection of 1 tumor cell in 5 million blood cells.

TABLE 13
Nested two-step headstart PCR detection of 1, 5, 50, or 1000
cancer cells per 1 million blood cells.

| Sample Name | Target Name | CT | GAPDH |
|---|---|---|---|
| L2 | FREM2 | 24.859 | 24.500 |
| PBMC + GSC 1000 | FREM2 | 30.984 | 24.734 |
| PBMC + GSC 50 | FREM2 | 35.298 | 25.676 |
| PBMC + GSC 5 | FREM2 | 33.655 | 25.487 |
| PBMC + GSC 1 | FREM2 | 37.227 | 23.361 |
| PMBC | FREM2 | undetermined | 24.330 |

In some embodiments, a third, nested primer pair, primer pair C, is used, nested three step headstart PCR. Primer pair C amplifies a region of DNA contained within the primer pair B amplification product. Various number of cycles can be used in each round. An exemplary protocol, for use of three primer pairs is: 10 cycles PCR amplification using primer pair A (first PCR reaction), followed by 10 cycles PCR amplification using primer pair B (second PCR reaction), followed by 30 cycles PCR amplification using primer pair C (third PCR reaction). The amplification product using primer pair A is used as input for amplification using primer pair B and the amplification product using primer pair B is used as input for amplification using primer pair C. In other embodiments, the first PCR reaction is 5-15 cycles, the second PCR reaction is 5-15 cycles, and the third PCR reaction is 20-40 cycles.

Nested multistep PCR can be used to detect GCSs in blood. Nested multistep PCR can be used to detect FREM2 as described above. Nested multistep PCR can also be used to detect SALL1, ID4, or NES using nested primers specific for these target genes. In some embodiments, nested multistep PCR is used to detect two or more of FREM2, SALL1, ID4, and NES. In some embodiments, nested multistep PCR is used to detect three or more of FREM2, SALL1, ID4, and NES. In some embodiments, nested multistep PCR is used to detect FREM2, SALL1, ID4, and NES. Nested multistep PCR to detect two or more genes can be performed in a single multiplex reaction or in separate uniplex reactions.

In addition to the four genes described above, nested multistep PCR can be used to detect other genes in the GCS regulatory network or downstream genes in other cancer master regulator networks. Using genetic analyses, the top 20 genes in a cancer of interest regulatory network, such as the GSC regulatory network, with the highest differential expression between the cancer cell, such as GSC, and PBMC are identified. Detection of the top 20 genes is performed in subjects with known GBM and healthy control subjects. Statistical methods are then used to assign weight to each of the 20 factors and develop an algorithm of scoring. A correlation between the strength of the scoring system and tumor burden and survival in patients is used as a GBM disease assessment and treatment response monitoring method. The described tests can be used to screen for cancer in apparently healthy or at risk subjects. The described tests can also be used to monitor the disease in cancer patients.

In some embodiments are described methods of assessing cancer risk comprising: using a statistical analysis to identify the top 20 genes differentially expressed in subjects having a cancer of interest; obtaining or having obtained a sample from a patient having, suspected of having, or at risk of developing in the cancer of interest; measuring, or having measured, expression of a plurality of the top 20 genes in the sample; assessing tumor burden or cancer risk based on the expression of the plurality of the top 20 genes in the sample.

Many modifications and other embodiments of the inventions set forth herein will come to mind to one skilled in the art to which the inventions pertain having the benefit of the teachings presented in the foregoing descriptions and the associated drawings. Therefore, it is to be understood that the inventions are not to be limited to the specific embodiments disclosed and that modifications and other embodiments are intended to be included within the scope of the appended claims. Although specific terms are employed herein, they are used in a generic and descriptive sense only and not for purposes of limitation.

What is claimed is:

1. A method of diagnosing a subject with a cancer, measuring in a liquid biopsy of said subject, the level of unmethylated DNA in a promoter and/or regulatory region of one or more master regulator genes of the cancer and comparing the results with a healthy reference sample, wherein a higher amount of unmethylated DNA of the master regulator genes detected in the liquid biopsy from the subject relative to the healthy reference sample is an indication of the cancer.

2. The method of claim 1, further comprising identifying the one or more master regulator genes of the cancer before the detecting step.

3. The method of claim 2, wherein the master regulator genes of the cancer are identified using GeneRep/nSCORE.

4. The method of any preceding claim, wherein the unmethylated DNA is detected using ATAC-seq.

5. The method of any preceding claim, wherein the unmethylated DNA is detected using a method comprising: CRISPR, digestion based assay followed by PCR, methylation specific PCR, E-ice-COLD-PCR, bead array analysis, pyrosequencing, PCR with high resolution melting, bisulfite sequencing.

6. A method of diagnosing a subject with a cancer, comprising measuring the expression level of one or more downstream targets of one or more master regulator genes of the cancer in a liquid biopsy of the subject and comparing the results with a healthy reference sample, wherein a higher expression level of downstream targets in the liquid biopsy from the subject relative to the healthy reference sample is an indication of the cancer.

7. The method of claim 6, further comprising identifying the one or more master regulators genes of the cancer and/or the one or more downstream targets of the master regulator genes before the measuring step.

8. The method of claim 6, wherein the cancer is GBM and the downstream targets are select from the group consisting of SALL1, ID4, FREM2, NES, MLXIPL, NKX2-2, KCNIP3, HLF, DDN, BATF2, MEOX2, OLIG2, PARGC1B, ACTN2, OTP, PRKCB, HOXA13, MNX1, ATOH7, RXRG, HOXA11, HOXD13, PEG3, RPH3A, HOXD3, CEBPB, ZNF248, BHLHE40, NMI, POU4F1, THRB, ENSG00000277459, FOXG1, FJX1, TUBB2B, MSI1, FKBP10, CBARP, GNG12, CD276, FAT1, CTXN1, DPYSL5, TYRO3, GJC1, CHST3, LRP4, PHF21B, ADAMTS9, CSPG5, TEAD1, BCAR1, EML1, RBFOX2, MPDZ, MSX1, EFNB3, ENAH, LYPD6, GTF2IRD1, TBX2, ANK2, C1QL1, ZIC1, EMP2, TMEM132A, CX3CL1, SYDE1, SLC16A2, SOX9, RND3, LARP6, CNN3, SPRY4, DPF1, PCGF2, BOC, OBSL1, SOX2, EPB41L1, MEX3A, NCS1, SMO, TMC7, SEZ6L2, POU3F2, FAM171A2, DENND2A, TANC1, PROX1, ENSG00000268592, PNPLA3, TSKU, DLX1, KIAA1549, MTSS1L, PTPRF, IRX5, DZIP1, MAGI1, ADCY6, BCHE, CXADR, TEAD4, PTPN21, CDR2L, ADGRL3, REEP2, SHROOM3, ARC, EEF1A2, ETV4, EGFR, UCHL1, KAZALD1, TJP1, ENSG00000237004, ETV5, CKB, KCNF1, MAP1B, S100A16, COL27A1, VGF, ALDH7A1, GAS1, LOC101927480, CITED1, ETV1, NOVA1, JPH1, FBXO17, CNKSR3, PDXP, PLEKHH3, PYGO1, SCARA3, RTL8B, LAMB1, MYH14, CASKIN1, NLGN2, PACSIN3, CA12, ARHGAP39, LAMA4, ZNF462, NR2F1, CSRP2, ARHGEF25, GLI3, TMEFF1, MIDI, B4GALNT1, SH3D19, SV2A, VAX2, CASC10, CSPG4, SNAI2, ARSJ, FSCN1, KHDRBS3, RASSF8, SMARCA1, TNC, PIR, ANTXR1, PHLDB1, CASKIN2, LAMC1, PAX6, ASPHD1, MAPK12, CAVIN1, TSPAN6, SEMA6D, NDRG4, PRR36, PFN2, SOX21, SPTBN2, GPC1, NRSN2, AADAT, GNA11, TCEAL9, DDAH1, KIAA1549L, LGR4, MEX3D, TNKS1BP1, PIMREG, SCD, FRMD6, HUNK, TMEM136, LRRC49, ARNT2, ENSG00000259495, DOCK1, PTPRS, TTC23, PPFIBP1, LHX2, SIX4, MAPK8IP1, IGDCC3, DMRTA2, STXBP1, PTPN14, SLC2A10, ARMC9, TBC1D16, FLNC, RHPN2, RHBDF1, P3H4, ENSG00000261578, DTNA, CELSR2, NOVA2, GPR176, VPS37D, SLC26A10, DNAJC22, and ZNRF3.

9. A method of diagnosing cancer in a subject with a cancer comprising
(a) obtaining or having obtained a sample from the subject
(b) analyzing or having analyzed chromosomal accessibility and/or DNA methylation of one or more master regulator genes of the cancer; and
(c) comparing the chromosomal accessibility and/or DNA methylation of the one or more master regulator genes of the cancer in the sample with the chromosomal accessibility and/or DNA methylation of the one or more master regulator genes of the cancer of a healthy reference sample, wherein an increase in chromosomal accessibility or a decrease in DNA methylation of a promotor and/or regulatory region of the master regulator genes detected in the sample relative to the healthy reference sample is an indication of the cancer.

10. The method of claim 9, wherein the sample is a liquid sample.

11. The method of claim 10, wherein the liquid sample is a blood sample.

12. The method of any one of claims 9-11, wherein chromosomal accessibility is analyzed by analyzing DNA methylation.

13. The method of claim 12, wherein analyzing chromosomal accessibility and/or DNA methylation comprises ATAC-seq, CRISPR, DNAse-seq, MNase-seq, a digestion based assay followed by PCR, methylation specific PCR, E-ice-COLD-PCR, Bead array analysis, pyrosequencing, PCR with high resolution melting, bisulfite sequencing.

14. The method any one of claims 9-13, wherein the cancer is GBM and the master regulator genes are selected from the group consisting of: TBX2, NKX2-2, BATF2, OLIG2, OTP, SALL1, ID4, FREM2, NES, MLXIPL, KCNIP3, HLF, DDN, MEOX2, PARGC1B, ACTN2, PRKCB, HOXA13, MNX1, ATOH7, RXRG, HOXA11, HOXD13, PEG3, RPH3A, HOXD3, CEBPB, ZNF248, BHLHE40, NMI, POU4F1, THRB, ENSG00000277459, FOXG1, FJX1, TUBB2B, MSI1, FKBP10, CBARP, GNG12, CD276, FAT1, CTXN1, DPYSL5, TYRO3, GJC1, CHST3, LRP4, PHF21B, ADAMTS9, CSPG5, TEAD1, BCAR1, EML1, RBFOX2, MPDZ, MSX1, EFNB3, ENAH, LYPD6, GTF2IRD1, ANK2, C1QL1, ZIC1, EMP2, TMEM132A, CX3CL1, SYDE1, SLC16A2, SOX9, RND3, LARP6, CNN3, SPRY4, DPF1, PCGF2, BOC, OBSL1, SOX2, EPB41L1, MEX3A, NCS1, SMO, TMC7, SEZ6L2, POU3F2, FAM171A2, DENND2A, TANC1, PROX1, ENSG00000268592, PNPLA3, TSKU, DLX1, KIAA1549, MTSS1L, PTPRF, IRX5, DZIP1, MAGI1, ADCY6, BCHE, CXADR, TEAD4, PTPN21, CDR2L, ADGRL3, REEP2, SHROOM3, ARC, EEF1A2, ETV4, EGFR, UCHL1, KAZALD1, TJP1, ENSG00000237004, ETV5, CKB, KCNF1, MAP1B, S100A16, COL27A1, VGF, ALDH7A1, GAS1, LOC101927480, CITED1, ETV1, NOVA1, JPH1, FBXO17, CNKSR3, PDXP, PLEKHH3, PYGO1, SCARA3, RTL8B, LAMB1, MYH14, CASKIN1, NLGN2, PACSIN3, CA12, ARHGAP39, LAMA4, ZNF462, NR2F1, CSRP2, ARHGEF25, GLI3, TMEFF1, MIDI, B4GALNT1, SH3D19, SV2A, VAX2, CASC10, CSPG4, SNAI2, ARSJ, FSCN1, KHDRBS3, RASSF8, SMARCA1, TNC, PIR, ANTXR1, PHLDB1, CASKIN2, LAMC1, PAX6, ASPHD1, MAPK12, CAVIN1, TSPAN6, SEMA6D, NDRG4, PRR36, PFN2, SOX21, SPTBN2, GPC1, NRSN2, AADAT, GNA11, TCEAL9, DDAH1, KIAA1549L, LGR4, MEX3D, TNKS1BP1, PIMREG, SCD, FRMD6, HUNK, TMEM136, LRRC49, ARNT2, ENSG00000259495, DOCK1, PTPRS, TTC23, PPFIBP1, LHX2, SIX4, MAPK8IP1, IGDCC3, DMRTA2, STXBP1, PTPN14, SLC2A10, ARMC9, TBC1D16, FLNC, RHPN2, RHBDF1, P3H4, ENSG00000261578, DTNA, CELSR2, NOVA2, GPR176, VPS37D, SLC26A10, DNAJC22, and ZNRF3

15. A method of diagnosing cancer in a subject with a cancer comprising
(a) identifying or having identified one or more genes in a master regulator network downstream of a master regulator of the cancer,
(b) obtaining or having obtained a sample from the subject,
(c) analyzing or having analyzed expression of the one or more genes; and
(d) comparing the expression level of the one or more genes in the sample with the expression level of the one or more genes of a healthy reference sample, wherein a differential expression of the one or more genes in the sample relative to the healthy reference sample is an indication of the cancer.

16. The method of claim 15, further comprising identifying or having identified at least one master regulator of the cancer before step (a).

17. The method of claim 16, where identifying one or more master regulators of the cancer comprises using GeneRep/nSCORE.

18. The method of any one of claims 15-17, wherein the sample is a liquid sample.

19. The method of claim 18, wherein the liquid sample is a blood sample.

20. The method of any one of claims 15-19, wherein the expression level is analyzed by PCR.

21. The method of claim 20, wherein the PCT comprises multi-step headstart PCR.

22. The method of any one of claims 15-21, wherein identifying or having identified one or more genes in a master regulator network downstream of a master regulator of the cancer comprises identifying or having identified the top 20 gene that are differentially expressed between the cancer and peripheral blood mononuclear cells.
